(19) United States
(12) Patent Application Publication
(10) Pub. No.: US 2007/0166320 A1
(43) Pub. Date: Jul. 19, 2007

(54) Agent for preventing/ameliorating diabetes and functional food for preventing/ameliorating diabetes

(76) Inventors: Kazuo Yamazaki, Saitama (JP); Eiichi Nakagawa, Saitama (JP)

Correspondence Address: MORGAN & FINNEGAN, L.L.P., 3 WORLD FINANCIAL CENTER, NEW YORK, NY, 10281-2101, US

(21) Appl. No.: 11/573,565

(22) PCT Filed: Aug. 15, 2005
(86) PCT No.: PCT/JP05/14908

§ 371 Date: Mar. 28, 2007

(30) Foreign Application Priority Data
Aug. 13, 2004 (JP) 2004-235913

(51) Int. Cl. A61K 36/06; A61K 36/16; A61K 36/906; A61K 36/45; A61K 33/32

(52) U.S. Cl. 424/195.15; 424/641; 424/702; 424/732; 424/752; 424/756; 424/774; 424/776; 514/54; 514/55; 514/57; 514/440

(57) ABSTRACT

It is an object of the present invention to provide an agent for preventing/ameliorating diabetes mellitus in which the agent has an effect of suppressing an increase in blood glucose level, and to provide a functional food containing the agent for preventing/ameliorating diabetes mellitus and having an effect of preventing/ameliorating diabetes mellitus. A dry powder, a dried substance, or a hot-water extract solution of a dry powder of dried tea tree mushroom (Agrocybe aegerita (Brig.) Sing) fruit body, which has been artificially cultivated in a fungus bed, is administered. A functional food of preventing/ameliorating diabetes mellitus is prepared by using, as part of food raw materials, a dry powder or a dried substance of dried tea tree mushroom fruit body, or a hot-water extract solution of the dry powder.

### TECHNICAL FIELD

The present invention relates to agents for preventing/ameliorating diabetes mellitus and relates to functional foods having an effect of preventing/ameliorating diabetes mellitus. The agent and the functional food contain a dried substance of tea tree mushroom (botanical name: Agrocybe aegerita (Brig.) Sing) that grows naturally on a tea tree or a processed substance thereof as an effective component.

### BACKGROUND ART

Recently, the number of people suffering from lifestyle-related diseases is increasing because of various factors such as a change in lifestyle, e.g., lack of exercise, a change in diet to meat-oriented one, and a change in eating habits to precooked food and fast food. Particularly, diabetes mellitus is a representative lifestyle-related disease. Diabetes mellitus is a syndrome having chronic hyperglycemia due to lack of insulin action as a main symptom and being associated with various metabolic disorders. Diabetes mellitus is roughly divided into insulin-dependent diabetes mellitus (type I diabetes) and non-insulin-dependent diabetes mellitus (type II diabetes), and is known to be a risk factor causing cerebral apoplexy and cardiopathy. In addition, a decrease in quality of life due to complications such as neuropathy, retinopathy, and renal impairment is a severe problem. The number of diabetes patients is estimated to be not less than 10% of the total population.

With respect to diabetes mellitus of which number is thus growing steadily, burdens on patients are thought to be reduced in various aspects if natural food substances not synthetic drugs can be routinely ingested. Conventionally, ingestion of so-called crude drugs has been known. Crude drugs are obtained from plants as raw materials and are ingested by brewing or extracting raw plants or dried plants. Recently, methods for efficiently manufacturing extract in industrial scale from plants or mushroom which are supplied for health maintenance are disclosed (for example, see Patent Document 1). In addition, some specific plants or mushrooms are known to be effective for preventing/ameliorating diabetes mellitus. For example, it has been found the fact that an alcohol extract of a tuberous root, leaves, or stem of Dioscorea bulbifera contains a substance for reducing blood glucose level (for example, see Patent Document 2). In addition, it is reported a functional food suppressing an increase in blood glucose level and containing an extract of the whole fruit (fruit containing fruit skin) of a dwarf citrus, which is a hybrid of Chinese orange and mandarin orange belonging to orange genus of the orange family and being natives to Philippine and southern China and broadly cultivated in Southeast Asia (for example, see Patent Document 3).

Further, an agent for reducing blood glucose level in which the agent contains an extract of Agaricus blazei, a type of mushroom, as an effective component is disclosed, and an agent for controlling blood glucose level in which the agent contains a cellulase-treated substance of Agaricus as an effective component is disclosed (for example, see Patent Documents 4 and 5). Similarly, an agent for preventing/ameliorating diabetes mellitus in which the agent contains a mushroom fruit body or a processed substance thereof of Climacodontaceae, a type of mushroom, as an effective component is disclosed (for example, see Patent Document 6). Furthermore, it is disclosed that a health food containing flour as a main material and blended with Hericium erinaceum as an essential material exhibits an excellent effect of reducing blood glucose level (for example, see Patent Document 7).

In addition, it is disclosed that tea tree mushroom highly contains a flavor component and an immunofunctional substance, and further a method for manufacturing a tea tree mushroom extract powder by extracting tea tree mushroom with hot water having a temperature of 90° C. or more at normal pressure over 1 to 1.2 hr, subjecting the extract to solid-liquid separation, controlling the solid content to 30% or less by concentration at 55° C. or less under reduced pressure, sterilizing the concentrated solution by heating, and drying the solution by spraying (for example, see Patent Document 8).

Patent Document 1: Japanese Laid-Open Patent Application No. 11-196818

Patent Document 2: Japanese Laid-Open Patent Application No. 2002-154978

Patent Document 3: Japanese Laid-Open Patent Application No. 2003-199527

Patent Document 4: Japanese Laid-Open Patent Application No. 11-80014

Patent Document 5: Japanese Laid-Open Patent Application No. 2004-155667

Patent Document 6: Japanese Laid-Open Patent Application No. 2002-187851

Patent Document 7: Japanese Laid-Open Patent Application No. 2000-166499

Patent Document 8: Japanese Laid-Open Patent Application No. 2004-65094

### DISCLOSURE OF THE INVENTION

Problem to be Solved by the Invention

It is an object of the present invention to provide an agent for preventing/ameliorating diabetes mellitus in which the agent has an effect of suppressing an increase in blood glucose level, and to provide a functional food containing the agent for preventing/ameliorating diabetes mellitus and having an effect of preventing/ameliorating diabetes mellitus.

Means to Solve the Problem

In order to solve the above-mentioned problems, the present inventors have focused on tea tree mushroom containing flavor which can be used as a seasoning and a medicinal component against cancer, etc. and have conducted intensive studies on searching for a new medicinal component and, as a result, have found a component having an effect of significantly suppressing an increase in blood glucose level. Thus, the present invention has been completed.

The present invention relates to (1) an agent for preventing/ameliorating diabetes mellitus in which the agent contains dried tea tree mushroom or a processed substance thereof as an effective component; (2) an agent for preventing/ameliorating diabetes mellitus, wherein the agent contains dried tea tree mushroom or a processed substance thereof and rafuma (yanlong tea); (3) the agent for preventing/ameliorating diabetes mellitus according to the above (1) or (2), wherein the agent further contains at least one component selected from the group consisting of chitosan, water-soluble chitosan, cellulose, hemicellulose, indigestible dextrin, lignin, agar, pectin, glucomannan, sodium alginate, α-lipoic acid, coenzyme Q10, vitamin E, vitamin C, ginkgo leaves, mulberry leaves, β-carotene, lycopene, lutein, astaxanthin, β-cryptoxanthin, catechin, turmeric, blueberry, cranberry, Japanese basil seeds, vitamin B₂, zinc, selenium, and a papaya extract powder; (4) the agent for preventing/ameliorating diabetes mellitus according to any one of the above (1) to (3), wherein the processed substance is a dried tea tree mushroom powder; (5) the agent for preventing/ameliorating diabetes mellitus according to any one of the above (1) to (3), wherein the processed substance is a capsule, a tablet, or granules of a dried tea tree mushroom powder; and (6) the agent for preventing/ameliorating diabetes mellitus according to any one of the above (1) to (3), wherein the processed substance is a hot-water extract solution of dried tea tree mushroom or a dry powder thereof.

Further, the present invention relates to (7) the agent for preventing/ameliorating diabetes mellitus according to any one of the above (1) to (3), wherein the processed substance is a dried extract of a hot-water extract solution of dried tea tree mushroom or a dry powder thereof; (8) the agent for preventing/ameliorating diabetes mellitus according to any one of the above (1) to (3), wherein the processed substance is a capsule, a tablet, or granules of a dried extract of a hot-water extract solution of dried tea tree mushroom or a dry powder thereof; (9) a functional food or food material for preventing/ameliorating diabetes mellitus, wherein the functional food or food material contains dried tea tree mushroom or a processed substance thereof; (10) a functional food or food material for preventing/ameliorating diabetes mellitus, wherein the functional food or food material contains dried tea tree mushroom or a processed substance thereof and rafuma (yanlong tea); (11) the functional food or food material for preventing/ameliorating diabetes mellitus according to the above (9) or (10), wherein the functional food or food material further contains at least one component selected from the group consisting of chitosan, water-soluble chitosan, cellulose, hemicellulose, indigestible dextrin, lignin, agar, pectin, glucomannan, sodium alginate, α-lipoic acid, coenzyme Q10, vitamin E, vitamin C, ginkgo leaves, mulberry leaves, β-carotene, lycopene, lutein, astaxanthin, β-cryptoxanthin, catechin, turmeric, blueberry, cranberry, Japanese basil seeds, vitamin B₂, zinc, selenium, and a papaya extract powder; (12) the functional food or food material according to the above (9) or (10), wherein the processed substance is a dried tea tree mushroom powder; (13) the functional food or food material according to the above (9) or (10), wherein the processed substance is a hot-water extract solution of dried tea tree mushroom or a dry powder thereof; (14) the functional food or food material according to the above (9) or (10), wherein the processed substance is a dried extract of a hot-water extract solution of dried tea tree mushroom or a dry powder thereof; (15) the functional food or food material according to the above (9) or (10), wherein the processed substance is a french-fry of dried tea tree mushroom; (16) the functional food or food material according to any one of the above (9) to (14), wherein the functional food or food material is tea, yogurt, drinkable yogurt, juice, milk, soymilk, alcoholic drinks, coffee, or a sports drink; and (17) the functional food or food material according to any one of the above (9) to (14), wherein the functional food or food material is bread or confectionery including baked goods such as pudding, crackers, biscuits, hardtack, cookies, bread, cake, jelly, and rice crackers, Japanese confectionery such as adzuki-bean jelly, frozen dessert, and chewing gum; noodles such as Japanese wheat noodles and buckwheat noodles; fish cakes such as kamaboko (steamed fish paste), ham, and fish meat sausage; a seasoning such as miso (soybean paste), soy sauce, dressing, mayonnaise, and sweetening; a dairy product such as cheese and butter; bean curd; konjac food (alimentary yam paste); a food boiled in soy sauce; gyoza (steam-baked meat pie); croquette; or a salad.

### BRIEF DESCRIPTION OF DRAWINGS

FIG. 1 shows an example of a method for preparing dried tea tree mushroom used in the present invention;

FIG. 2 is a graph showing (fasting) blood glucose levels measured before and during (June 2nd or later) administration of tea tree mushroom according to the present invention;

FIG. 3 is a graph showing HbA1c levels measured before and during (June 2nd or later) administration of tea tree mushroom according to the present invention;

FIG. 4 is a graph showing (fasting) blood glucose levels measured before and during (June 13th or later) administration of tea tree mushroom according to the present invention;

FIG. 5 is a graph showing HbA1c levels measured before and during (June 13th or later) administration of tea tree mushroom according to the present invention;

FIG. 6 is a graph showing changes in blood glucose levels after glucose load (after administration of various types of test solutions) in a test group of the present invention, a glucose control group, and a product control group; and

FIG. 7 is a graph showing calculated glucose AUC after glucose load (after administration of various types of test solutions) in a test group of the present invention, a glucose control group, and a product control group.

### BEST MODE OF CARRYING OUT THE INVENTION

Tea tree mushroom which grows naturally on tea trees in neighborhood of Ibu-san, Fukken-sho, China characteristically has a thin pileus and a long stem. The flavor is based on a large amount of amino acids contained therein. Tea tree mushroom contains glutamic acid about 4 times higher than that of shuitake mushroom and about 10 times higher than that of tangle weed, and contains aspartic acid about 40 times higher than that of shuitake mushroom. Tea tree mushroom contains all 18 kinds of amino acids and, particularly, contains large amounts of essential amino acids. Table 1 shows contents (mg) of various amino acids in 100 g of each dry powder. The data are results of analysis performed at the Japan Food Research Laboratories. Table 2 shows contents of vitamins contained in a dried tea tree mushroom powder. The data are results of analysis performed at the Japan Food Research Laboratories. Thus, it is confirmed that the powder abundantly contains vitamin B group, which is effective in relieving fatigue. In addition, the results of analysis at the Japan Food Research Laboratories show that a dried tea tree mushroom powder contains about 20% β-glucan, which is famous as an effective component of such as Agaricus blazei (which contains about 10% β-glucan), and abundantly contains dietary fiber, as shown in Table 3.

TABLE 1

| | Tea tree | Shiitake | |
|---|---|---|---|
| Amino acid component | mushroom | mushroom | Kombu |
| Arginine | 1070 | 280 | 6 |
| Lysine | 1150 | 170 | 5 |
| Histidine | 510 | 79 | 3 |
| Phenylalanine | 790 | 71 | 6 |
| Tyrosine | 550 | 42 | 3 |
| Leucine | 1410 | 46 | 6 |
| Isoleucine | 880 | 43 | 4 |
| Methionine | 240 | 13 | 1 |
| Valine | 1170 | 79 | 8 |
| Alanine | 1290 | 160 | 100 |
| Glycine | 950 | 61 | 5 |
| Proline | 780 | 33 | 53 |
| Glutamic acid | 3050 | 730 | 310 |
| Serine | 1060 | 92 | 19 |
| Threonine | 1050 | 850 | 34 |
| Aspartic acid | 1910 | 47 | 58 |
| Tryptophan | 300 | 11 | 0 |
| Cystine | 230 | 59 | 2 |

TABLE 2

| Vitamin B Group | Content |
|---|---|
| Thiamin (VB1) | 0.84 mg/100 g |
| Riboflavin (VB2) | 2.44 mg/100 g |
| Vitamin B6 | 0.46 mg/100 g |
| Vitamin B12 | Not detected |
| Folic acid | 0.28 mg/100 g |
| Pantothenic acid | 7.54 mg/100 g |

TABLE 3

| Dietary Fiber | Content |
|---|---|
| Water-soluble dietary fiber | 1.7 g/100 g |
| Insoluble dietary fiber | 31.4 g/100 g |
| Total | 33.1 g/100 g |

The agent for preventing/ameliorating diabetes mellitus according to the present invention is not specifically limited as long as the agent contains dried tea tree mushroom or a processed substance thereof as an effective component. The functional food or food material for preventing/ameliorating diabetes mellitus (namely, a functional food or food material which is provided with an indication to be used for preventing/ameliorating diabetes mellitus) according to the present invention is not specifically limited as long as the functional food or food material contains dried tea tree mushroom or a processed substance thereof. The agent for preventing/ameliorating diabetes mellitus and the functional food or food material for preventing/ameliorating diabetes mellitus according to the present invention can be advantageously used as an agent for preventing/treating diabetes mellitus or an agent for ameliorating the symptoms by suppressing such as an increase in blood glucose level.

The agent for preventing/ameliorating diabetes mellitus and the functional food or food material for preventing/ameliorating diabetes mellitus (namely, a functional food or food material which is provided with an indication to be used for preventing/ameliorating diabetes mellitus) according to the present invention may contain dried tea tree mushroom or a processed substance thereof and rafuma (yanlong tea). Such an agent or a functional food or food material has functions of suppressing an increase in blood glucose level and reducing the absorption of glucose. Therefore, for example, the agent and the functional food or food material according to the present invention can be advantageously used as an agent for preventing/treating diabetes mellitus or an agent for ameliorating the symptoms thereof or further used for preventing lifestyle-related diseases without adverse effects such as hypoglycemia.

The agent for preventing/ameliorating diabetes mellitus and the functional food or food material for preventing/ameliorating diabetes mellitus (namely, a functional food or food material which is provided with an indication to be used for preventing/ameliorating diabetes mellitus) according to the present invention may further contain at least one component selected from the group consisting of chitosan, water-soluble chitosan, cellulose, hemicellulose, indigestible dextrin, lignin, agar, pectin, glucomannan, sodium alginate, α-lipoic acid, coenzyme Q10, vitamin E, vitamin C, ginkgo leaves, mulberry leaves, β-carotene, lycopene, lutein, astaxanthin, β-cryptoxanthin, catechin, turmeric, blueberry, cranberry, Japanese basil seeds, vitamin B₂, zinc, selenium, and a papaya extract powder. By mixing the dietary fiber, an anti-oxidant, a vitamin, a mineral, or an enzyme to the agent or the functional food or food material, it can be expected to significantly enhance the effect of tea tree mushroom for suppressing an increase in blood glucose level.

Tea tree mushroom used in the present invention may be a fruit body of a wild tea tree mushroom growing naturally in the wild or a fruit body of an artificially cultivated tea tree mushroom. However, the product amount of the wild tea tree mushroom is small, and further a picked tea tree mushroom has a shelf life of only one week and is fragile so that the pileus is easily fallen apart from its stem by touching it. Therefore, a fruit body of an artificially cultivated tea tree mushroom is preferable because of its stability in quality such as component content. The method of the artificial cultivation is not specifically limited, but a fungus bed cultivation method allows harvesting tea tree mushroom having a stable quality, such as component contents, inexpensively and stably all the year round and is more preferable than a method using a wild tea tree. Here, the term “fungus bed cultivation method” means a method of seeding fungus onto a material composed of water-holding material and a nutrition source, without using a log, and cultivating the fungus under conditions of controlled temperature, humidity, and illumination. The artificially cultivated tea tree mushroom in a dried state is confirmed to be safe by the results (see Table 4) of analysis on the residue of agricultural chemicals used on tea tree mushroom. The analysis was performed at the Japan Food Research Laboratories.

TABLE 4

| Type of agricultural | | Sensitivity | |
|---|---|---|---|
| chemical | Result | limit | Method |
| Dieldrin | Not | 0.01 ppm | Gas chromatography |
| | detected | | |
| Chlorpyrifos | Not | 0.01 ppm | Gas chromatography |
| | detected | | |
| Methamidophos | Not | 0.05 ppm | Liquid |
| | detected | | chromatography |
| Fenvalerate | Not | 0.02 ppm | Gas |
| | detected | | chromatography |

The dried tea tree mushroom used in the present invention can be obtained by drying the fruit body. The method of drying is not specifically limited as long as the method is used for drying similar kinds of mushroom. Specifically, the drying may be performed by solar drying, hot-air drying, or a combination of solar drying and hot-air drying, for example. The conditions for drying are not specifically limited. For example, the hot-air drying is preferably conducted by heating at a drying temperature of 40 to 90° C., in particular, 60 to 70° C. for 5 to 9 hr. By drying at this temperature range, the production of a burnt smell and the decomposition of an effective component contained in tea tree mushroom can be suppressed. In addition, examples of the processed substance of dried tea tree mushroom include a powder of a dried fruit body; a capsule, a tablet, or granules of this dry powder; a solvent-extract solution of a dried fruit body or a dry powder thereof; an extract in powder or solid form prepared by drying the solvent-extract solution by spray drying or lyophilization; a capsule, a tablet, or granules of this extract; an enzyme-processed substance such as a cellulase-processed substance of a dried fruit body or a dry powder; and fermented substance of a dried fruit body or a dry powder with such as lactic acid bacterium or yeast. Examples of a solvent used for preparation of a solvent-extract solution of a dried fruit body or a dry powder include normal-temperature water, hot water, hydrated ethanol, and ethanol. In particular, hot-water extraction is preferable. Further, as mentioned above, after the extraction, a powder may be obtained by concentration after solid-liquid separation and drying the concentrated solution by spraying. In addition, a solid substance may be obtained by lyophilization. Furthermore, the above-mentioned dry powder or spray-dried powder may be formed into granules, capsules, or tablets. From viewpoints of improvement in yield of a component in a fruit body which has an effect of suppressing an increase in blood glucose level present, prevention of inactivation of the component, and the pattern of use as a food material, a dry powder of a fruit body, a hot-water extract of a fruit body, or a dry powder obtained from the hot -water extract is preferably used. Furthermore, the processed substance of dried tea tree mushroom according to the present invention may be a substance obtained by french-frying dried tea tree mushroom at a temperature of 165 to 175° C. for 7 to 20 sec. The excessively adhered oil by frying can be more completely removed by the centrifugation performed immediately after the frying process. The shelf life of french-fry is long, and french-fry is delicious on its own and safe hygienically. In addition, a moderate amount of french-fry can be eaten at any time. Therefore, french-fry can be particularly used as a functional food or food material for preventing/ameliorating diabetes mellitus according to the present invention.

The agent for preventing/ameliorating diabetes mellitus according to the present invention can specifically decrease fasting blood glucose levels and has a function of preventing/ameliorating diabetes mellitus by suppressing an increase in blood glucose level. Therefore, the agent can be used as an agent for preventing diabetes mellitus or for ameliorating the symptoms and further can be advantageously used as a pharmacological composition material for making a food into a functional food having functions of preventing/ameliorating diabetes mellitus by adding/mixing the agent to the food. When the agent for preventing/ameliorating diabetes mellitus according to the present invention is used as a drug, pharmaceutically acceptable various kinds of additives generally used for preparing drugs, i.e., a carrier, a binder, a stabilizer, a filler, a diluent, pH buffer, a disintegrating agent, a solubilizer, a solubilizing agent, and a tonicity agent, may be added to the agent. The agent for preventing or ameliorating/treating diabetes mellitus can be orally or parenterally administered. That is to say, the agent can be orally administered in general administration form, for example, in powder, granule, capsule, syrup, or suspension form, or the agent may be parenterally administered in form such as a solution, emulsion, or suspension. However, oral administration is preferable. The administration dosage can be optionally controlled depending on symptoms, sex, and age. Generally, the effects of preventing/ameliorating diabetes mellitus can be achieved by ingesting 1 to 100 g, preferably 5 to 50 g, more preferably 10 to 30 g of the agent, in terms of a dried fruit body, per one day for an adult once or in two or three divided doses. In addition, the agent for preventing/ameliorating diabetes mellitus according to the present invention may be ingested in addition to a drug for diabetes mellitus which is prescribed by a physician.

The functional food or food material for preventing/ameliorating diabetes mellitus according to the present invention can be obtained by using the above-described agent for preventing/ameliorating diabetes mellitus as a part of raw materials of food and drink products or obtained by adding/mixing the agent to a food or drink during or after the manufacturing process. The functional food does not have any specific limitation. Examples of the functional food include confectionery including baked goods such as cookies, bread, biscuits, hardtack, cake, and rice crackers, tablet sweets such as soda-flavored candy, Japanese confectionery such as adzuki-bean jelly, frozen dessert such as pudding, jelly, and ice cream, chewing gum, and candy; snacks such as crackers and chips; noodles such as Japanese wheat noodles and buckwheat noodles; fish cakes such as kamaboko (steamed fish paste), ham, and fish meat sausage; dairy products such as cheese and butter; seasonings such as miso (soybean paste), soy sauce, dressing, mayonnaise, and sweetening; various types of prepared foods such as bean curd, konjac food (alimentary yam paste), a food boiled in soy sauce, gyoza (steam-baked meat pie), croquette, salads, and stews; and various types of drinks such as tea, yogurt, drinkable yogurt, juice, milk, soymilk, alcoholic drinks, coffee, black tea, boiled tea, oolong tea, and sports drinks. For example, a dried product of tea tree mushroom fruit body is micronized into a fine powder, and the resulting fine powder may be made into tablets according to an ordinary method to produce a tablet sweet. In this case, the fine powder may be granulated and then made into tablets. In addition, a dried product of tea tree mushroom fruit body is micronized into a fine powder, and the resulting fine powder may be mixed with lactose, dextrin, and dried yeast and made into tablets.

The present invention will now be described more specifically with reference to examples, but the technical scope of the present invention is not limited to these examples.

### EXAMPLE 1

(Preparation of Dried Tea Tree Mushroom)

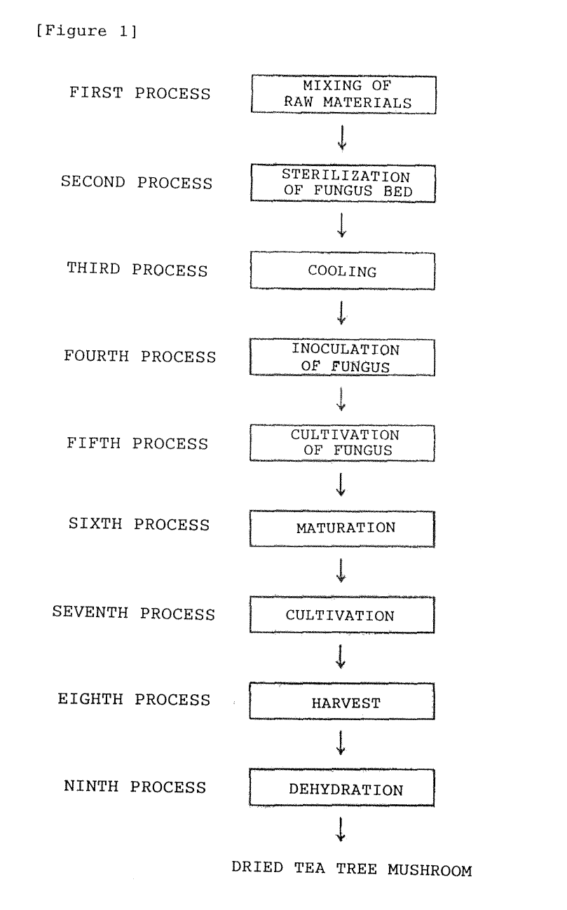

FIG. 1 schematically shows a manufacturing process of dried tea tree mushroom. The process will be described with reference to FIG. 1. In a first process (mixing of raw materials), a water-holding material such as sawdust is mixed with a culture source containing a nutrition source such as bran for cultivation. In a second process (sterilization of fungus bed), the mixed culturing medium, i.e., a fungus bed, is sterilized by a sterilizing means such as autoclaving. In a third process (cooling), the fungus bed is cooled by being left at room temperature for about 48 hr. In a fourth process (inoculation of fungus), tea tree mushroom fungus is inoculated in an inoculation chamber. In a fifth process (cultivation of fungus), cultivation (culturing) is conducted at 18 to 25° C. for 35 to 45 days. In a sixth process (maturation), aging is conducted in the cultivation chamber at 20 to 25° C. for 10 to 15 days. In a seventh process (cultivation), budding is conducted in the cultivation chamber set at a temperature of 18 to 22° C. and a humidity of 90 to 95% for 7 to 10 days. In an eighth process (harvest), the mushroom which has grown to have a pileus of 2 to 7 cm in size is harvested. In a ninth process (dehydration), dried tea tree mushroom is obtained by solar drying for one day and then hot-air drying at about 60° C. for 9 hr.

### EXAMPLE 2

### Treatment Example 1

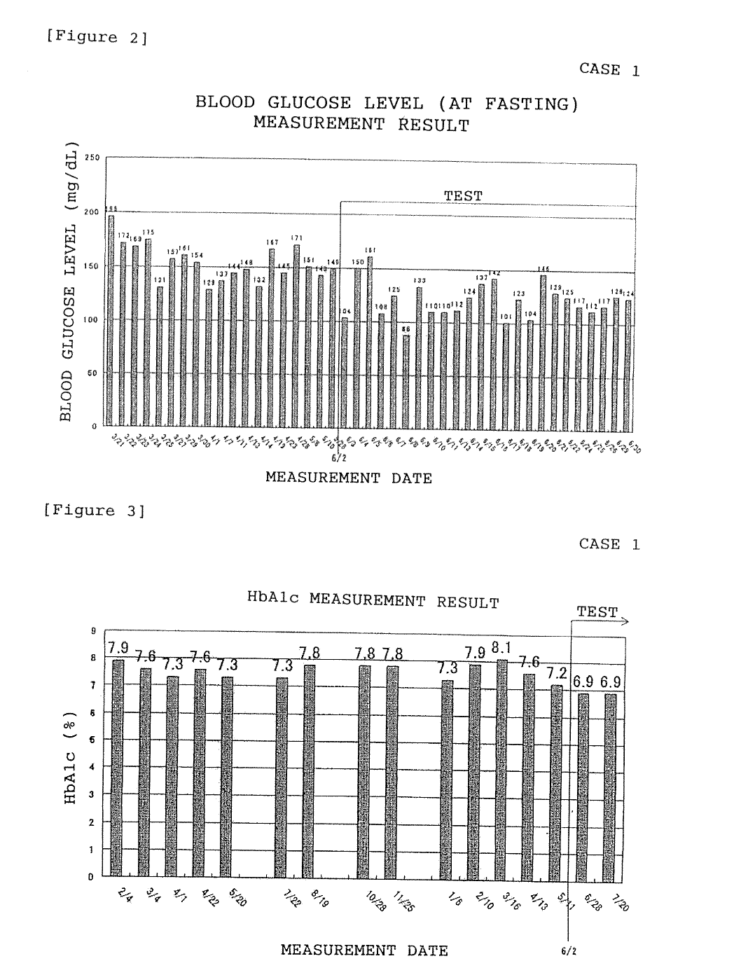

A 71-year old man was diagnosed to have diabetes mellitus in 1991 and, since then, has been treated with diet and exercise under guidance of a physician. As a drug for treatment of the diabetes, Euglcon (trade name, the general name is glibenclamide) has been administered at a dose of 1.25 mg/day since the year 1991, 1.88 mg/day since the year 2001, and 2.5 mg/day since the year 2003 till Jun. 3, 2004. On and after June 3rd, in addition to 2.5 mg/day of Euglcon, an extract which was extracted with about 150 cc of hot water from 5 g of dried tea tree mushroom of the present invention has been ingested twice a day in the morning and evening, with permission of the physician. FIG. 2 shows the (fasting) blood glucose levels since Mar. 21, 2004 till Jun. 30, 2004 (when the fasting blood glucose level per 1 dL of plasma is lower than 110 mg, it is determined to be “normal”; when the level is 126 mg or more, it is determined to be “diabetes”, and when the level is 110 mg or more and lower than 126 mg, it is determined to be “borderline diabetes”). FIG. 3 shows blood glucose predict hemoglobin A1c (HbA1c) levels measured once a month during nearly the same period. Hemoglobin A1c is formed by the binding of glucose to hemoglobin, which is a protein of erythrocytes. Though the fasting blood glucose level changes from day to day, the hemoglobin A1c level reflects the average blood glucose level over the preceding one to two months and, therefore, can be used as an indicator of blood glucose control.

As shown in FIG. 2, on and after June 3rd when tea tree mushroom according to the present invention was ingested, the number of day when the blood glucose level (mg/dl) was 100 or more was once, slightly higher than 100 was 4 times, and about 110 was 3 times. Since such a low level was not observed before the ingestion, it was confirmed that tea tree mushroom of the present invention had an effect of suppressing an increase in blood glucose level. In addition, as shown in FIG. 3, the hemoglobin A1c level was reduced to 6.9 after the ingestion of tea tree mushroom of the present invention. Since such a low level was not observed before the ingestion, it is also clear from this hemoglobin A1c level that the present invention has an effect of suppressing an increase in blood glucose level.

### EXAMPLE 3

### Treatment Example 2

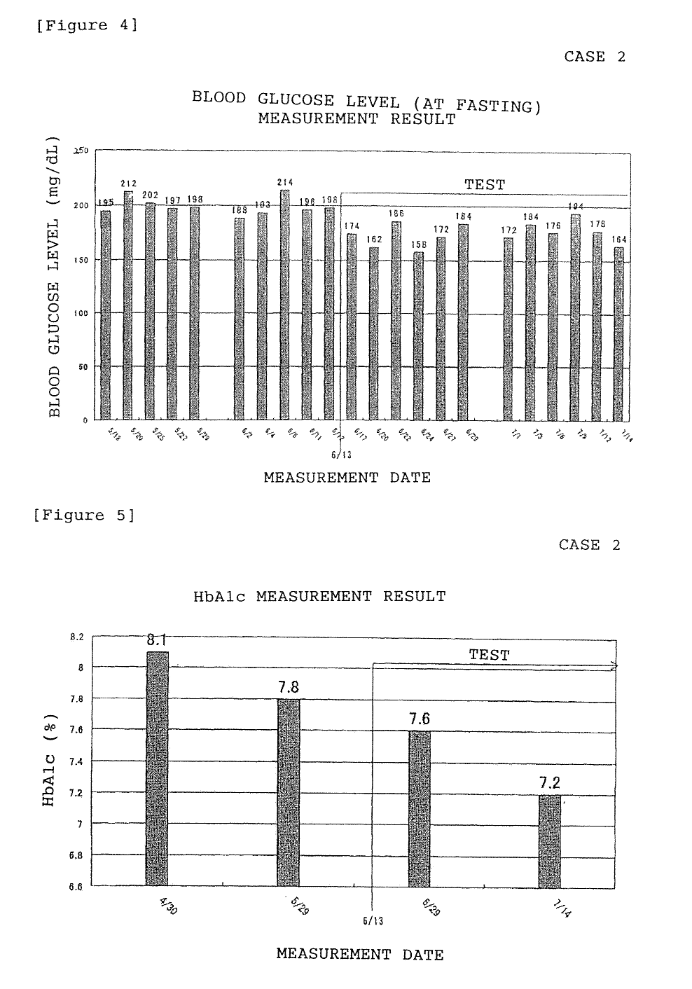

A 65-year old man suffering from diabetes mellitus has been treated for four months by injecting 0.8 ml/time of insulin three times per day around breakfast time, around lunch time, and around supper time under guidance of a physician. Recent two months, the insulin injection of the above-mentioned prescription, three times per day, has been continued, and on and after June 13th, in addition to the insulin injection, 2 g of a fine powder of dried tea tree mushroom has been ingested three times a day in the morning, daytime, and evening, with permission of the physician. FIG. 4 shows fasting blood glucose levels measured with a portable blood glucose meter over recent two months. FIG. 5 shows hemoglobin A1c (HbA1c) levels measured by an ambulant physician for three months. As shown in FIG. 4, it was observed that the blood glucose level after the start of the ingestion had an obvious tendency to be reduced compared to that before the ingestion. That is to say, though the blood glucose levels before the ingestion were about 200 mg/dl, those after the ingestion were lower than 200 mg/dl. Further, a level of near 150 mg/dl was observed. In addition, blood glucose predict hemoglobin A1c (HbA1c) levels, which reflect the average blood glucose level over one to two months before the test, were significantly improved after the ingestion. Thus, it is also clear from the hemoglobinA1c levels that the present invention has an effect of suppressing an increase in blood glucose level.

### EXAMPLE 4

(Manufacturing of Functional Snack Food)

Fifty parts by weight of water was added to 70 parts by weight of hard flour, 23 parts by weight of a tea tree mushroom fine powder, 7 parts by weight of a sprouted unpolished rice dry powder, 3 parts by weight of live yeast, 0.1 parts by weight of a yeast food, 5 parts by weight of gluten, 10 parts by weight of sugar, 1 part by weight of table salt, and 4 parts by weight of margarine. The resulting mixture was mixed with a vertical mixer for 20 min. Then, the resulting dough was fermented in a drier at 30° C. for 5 hr. Then, the dough was punched and molded, and then cut into quadrangles of 0.5 mm in thickness, 50 mm in length, and 50 mm in width. The quadrangular-formed dough of 50 mm×50 mm in size was provided with ten holes for releasing gas and was fired in an oven to obtain a cracker-like snack. This cracker-like snack is a dark brown crispy snack food and also is a portable and low calorie health snack food containing 20% of a tea tree mushroom powder and being excellent in storage stability.

### EXAMPLE 5

(Manufacturing of Functional Drink)

Tea tree mushroom was ground into a powder passing through 30 mesh. Water of 10 times volume was added thereto, and extraction was carried out at 98° C. for 30 min under heating and stirring. The pH of the extract was adjusted to 5.5, and 0.1 wt % of cellulase AP3 (manufactured by Amano Pharmaceutical Co., Ltd.) was added thereto for enzyme treatment at 45° C. for 4hr. Then, the reaction solution was heated to 90° C. to terminate the reaction. After the solid-liquid separation, the treatment solution was dried by spraying according to a general method to obtain a spray-dried product. This spray-dried product was a soluble brown powder being hygroscopic and having a slight sweet aroma and flavor.

Thirty parts by weight of green tea leaves were extracted with 900 parts by weight of water at 70° C. for 5 min. The tea-leaves were removed by rough filtration. Then, by suction filtration using filter paper, 700 parts by weight of green tea extract solution was obtained. To 300 parts by weight of this green tea extraction solution, 5 parts by weight of the above-mentioned spray-dried tea tree mushroom extract was added. The pH of the resulting mixture was adjusted to 5 to 6 by adding L-ascorbic acid and sodium hydrogen carbonate, and water was added thereto to make the total volume 1000 parts by weight. The resulting mixture was canned and sterilized at 120° C. for 10 min to obtain a green tea healthy drink after cooling. This green tea healthy drink has a characteristic flavor due to abundant amino acids derived from tea tree mushroom and a good taste in both when it is warmed and cooled. The green tea healthy drink is a healthy food for improving blood glucose level and can be continuously drunk without hardness, if necessary.

### EXAMPLE 6

(Manufacturing of Functional Candy)

To starch syrup and sugars (including white sugar, coarse sugar, and brown sugar) as candy raw materials, a required amount of water was added. The resulting mixture was heated to about 150° C. and stirred the whole mixture to make it into a starch syrup-like state. The starch syrup-like material was cooled to 70° C. or less. Then, during the material was still viscous, 30 parts by weight of a dried tea tree mushroom powder was sprinkled on the starch syrup-like material while mixing and kneading. The mushroom powder was approximately equally mixed with the whole material, and then the candy raw material was molded by putting the material into a desired-shaped cast so as to draw the candy raw material. After cooling, the material was demolded to form a drop-like shaped candy. Thus, a drop-like shaped candy was obtained.

### EXAMPLE 7

(Manufacturing of Functional Tea Tree Mushroom French-fry)

Dried tea tree mushroom (500 g) was arranged with interspaces in a wire woven basket so that the mushroom is not stuffed. The tea tree mushroom was fried by being immersed in an oil tank containing 36 L of vegetable oil heated to 170° C. for 10 sec. After the frying, the adhering excessive oil was removed by dropping, and 675 g of tea tree mushroom french-fry was obtained by cooling. This french-fry was a brown crunchy snack-like food having a good smell and taste and was favored by both adults and children.

As described above, it was obvious that the tea tree mushroom and the processed substance thereof according to the present invention can be used as an agent or a functional food or food material for preventing/ameliorating diabetes mellitus. Next, some examples of tea tree mushroom agent processed products were manufactured by combining the tea tree mushroom according to the present invention and various secondary components which were generally known as functional materials. Using one of the tea tree mushroom processed products as an example, the effect of suppressing an increase in blood glucose level was confirmed.

### EXAMPLE 8

Prior to the research on the effect of suppressing an increase in blood glucose level of a tea tree mushroom processed product containing secondary components, tea tree mushroom processed products were manufactured (see Table 5).

(Manufacturing of Tea Tree Mushroom Processed Product A)

The total 100 parts by weight of a tea tree mushroom processed product A was prepared by mixing 60 parts by weight of the dried tea tree mushroom powder obtained in Example 1 as an active component with 30 parts by weight of a powder of rafuma (yanlong tea) which was readily commercially available and 10 parts by weight of a papaya extract powder (manufactured by Koso Corporation) as secondary components (see Table 5A).

(Manufacturing of Tea Tree Mushroom Processed Product B)

The total 100 parts by weight of a tea tree mushroom processed product B was prepared by mixing 60 parts by weight of the dried tea tree mushroom powder obtained in Example 1 as an active component with 30 parts by weight of a water-soluble chitosan powder (manufactured by Kitosan Food Corporation), 9 parts by weight of turmeric which was readily commercially available, and 1 part by weight of vitamin C as secondary components (see Table 5B).

(Manufacturing of Tea Tree Mushroom Processed Product C)

The total 100 parts by weight of a tea tree mushroom processed product C was prepared by mixing 70 parts by weight of the dried tea tree mushroom powder obtained in Example 1 as an active component with 30 parts by weight of indigestible dextrin which was readily commercially available as a secondary component (see Table 5C).

TABLE 5
Mixing Ratios for Processed Tea Tree Mushroom Products

| | A (weight %) | B (weight %) | C (weight %) |
|---|---|---|---|
| Active ingredient | | | |
| Dried tea tree | 60 | 60 | 70 |
| mushroom | | | |
| Supplementary | | | |
| ingredient | | | |
| Water-soluble | | 30 | |
| chitosan | | | |
| Yanlong tea | 30 | | |
| Indigestible | | | 30 |
| dextran | | | |
| Papaya extract | 10 | | |
| powder | | | |
| Turmeric powder | | 9 | |
| Vitamin C | | 1 | |

### EXAMPLE 9

(Effect of Suppressing an Increase in Blood Glucose Level of Tea Tree Mushroom Processed Product)

The effect of suppressing an increase in blood glucose level of the tea tree mushroom processed product A manufactured in Example 8 was investigated by conducting an animal test. The process of the animal test and the results will now be shown. Further, this test was conducted with coordination of the Japan Food Research Laboratories.

(Tested Animal)

Six-week old ICR strain male mice were purchased from Japan SLC Inc. and were preliminarily fed for 6 days in order to confirm that the mice were normal under general conditions, and then used for the test. Each five test animals were fed in a polycarbonate gauge placed in a feeding room set to a room temperature of 23° C.±2° C. and an illumination time of 12 hr/day. Feed [feed, for mouse/rat/hamster, irradiated with gamma-ray; CRF-1 manufactured by Oriental Yeast Co., Ltd.] and drinkable water (tap water) were freely ingested.

(Specification of Group)

The mice were classified into a group (test group) in which mice were administered with the tea tree mushroom processed product A (hereinafter, referred to as product) manufactured in Example 8 and glucose, a group (glucose control group) in which the mice were administered with glucose only, and a group (product control group) in which the mice were administered with the product only. The number of mice in each group was 6 (see Table 6). The evaluation was conducted between the test group and the glucose control group. The product control group was used for confirming a change in blood glucose level caused by the administration of the product only.

TABLE 6
Specification of Group

| | | Administered | Administered |
|---|---|---|---|
| | Number of | amount of product | amount of glucose |
| Group | animal | (mg/kg) | (mg/kg) |
| Test group | 6 | 2000 | 2000 |
| Glucose | 6 | 0 | 2000 |
| control group | | | |
| Product | 6 | 2000 | 0 |
| control group | | | |

(Preparation of Test Solution)

Test solutions to be administered to the test group, the glucose control group, and the product control group were prepared so as to each contain 200 mg/mL of the product and/or glucose as shown below.

(1) Test Group

Water for injection was added to 4.00 g of the product and 4.00 g of glucose to obtain a total volume of 20 mL (the product concentration and the glucose concentration were each 200 mg/mL).

(2) Glucose Control Group

Water for injection was added to 4.00 g of glucose to obtain a total volume of 20 mL (glucose concentration: 200 mg/mL).

(3) Product Control Group

Water for injection was added to 4.00 g of the product to obtain a total volume of 20 mL (product concentration: 200 mg/mL).

(Glucose Tolerance Test)

Mice were fasted for 16 hr or more and measured for blood glucose levels and weights and grouped into the three groups not to cause variations in blood glucose level and weight among groups. The mice of each group were orally administered with 10 ml/kg of the respective test solutions once with a gastric tube. The glucose level of blood obtained by cutting the end of tail of each mouse was measured with ACCU-CHECK Active (manufactured by Roche Diagnostics) at 15, 30, 60, 90, 120, 240, and 300 min after the administration, assuming the time of the administration to be 0 min. However, the measurement of the blood glucose level was discontinued when the blood glucose level became lower than that at the administration.

(Evaluation)

The effect of suppressing an increase in blood glucose level caused by administration of the product was evaluated by comparing the maximum blood glucose levels of the test group and the glucose control group to determine whether or not an increase in blood glucose level was suppressed by the ingestion of the product. In addition, by using the glucose incremental area under curve (hereinafter, referred to as AUC) as an indicator of the amount of absorbed glucose, it was evaluated whether or not the absorption of glucose was suppressed by the ingestion of the product. Here, the term “glucose AUC” was defined as an area, in a graph plotted blood glucose level (mg/dL) in vertical axis and time (h) in lateral axis, surrounded by a line parallel to the time axis passing through the blood glucose level at the time of administration and a curve of a change in blood glucose level. However, the blood glucose levels at the time of discontinuation of the measurement were not used in the evaluation, and the results by 240 min in the test group, 120 min in the glucose control group, and by 60 min in the product control group were used.

(Test Result)

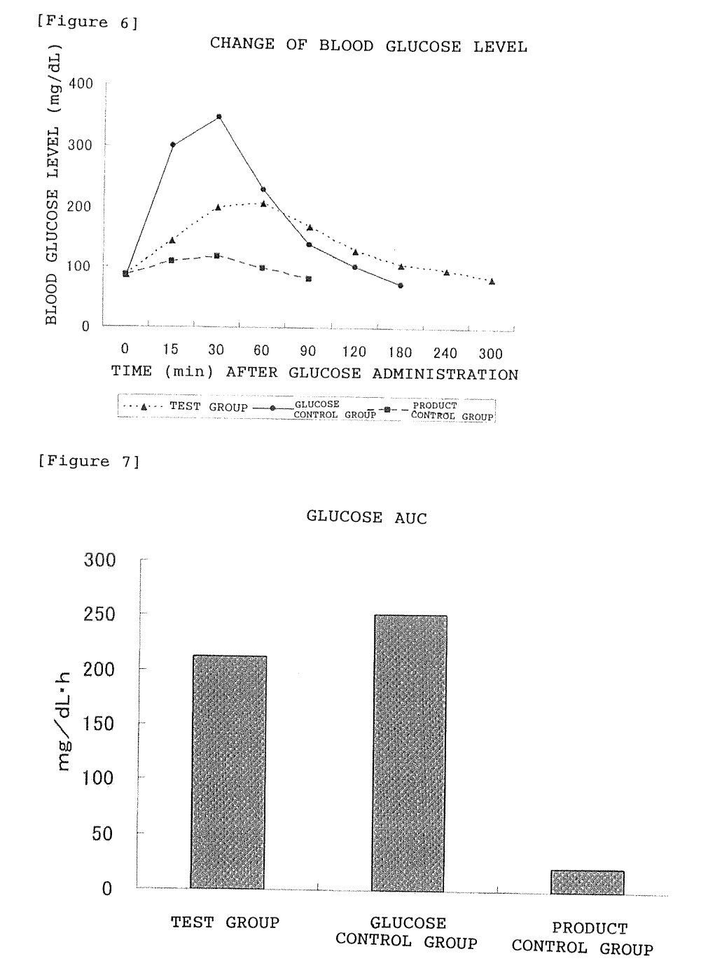

1. Comparison of the Maximum Blood Glucose Level (see Table 7 and FIG. 6)

In the glucose control group, the blood glucose level reached the maximum level (346.2±38.8 mg/dL) at 30 min after the administration and decreased to a level lower than that at the time of administration at 180 min after the administration.

On the other hand, in the test group, the blood glucose level reached the maximum level (206.3±51.5 mg/dL) at 60 min after the administration, but the level was significantly low compared to that in the glucose control group (p<0.05). Then, the level gradually decreased to a level lower than that at the time of administration at 300 min after the administration.

In the product control group, a slight increase in the blood glucose level was observed, but the level decreased to a level lower than that at the time of administration at 90 min after the administration. The results are shown as the average±standard deviation. Further, by a test, the level of significance adopted was not exceeding 5%.

TABLE 7
Change in Blood Glucose Level

| | Time (min) after Glucose Administration | | | | | | | | |
|---|---|---|---|---|---|---|---|---|---|
| | 0 | 15 | 30 | 60 | 90 | 120 | 180 | 240 | 300 |
| Test group | 86.5 | 143.3 | 198.8 | 206.3* | 168.3 | 127.3 | 106.3 | 96.5 | 83.8 |
| | 9.7 | 30.8 | 22 | 51.5 | 37.5 | 24 | 23.6 | 19.7 | 16.1 |
| Glucose | 86.5 | 300.3 | 346.2 | 228.5 | 138.5 | 101.7 | 72.3 | | |
| control group | 9.6 | 49.1 | 38.8 | 38 | 21.4 | 9.2 | 6.2 | | |
| Product | 85.8 | 109.8 | 118 | 98.2 | 80.7 | | | | |
| control group | 10.6 | 23.8 | 19.8 | 16.6 | 21.6 | | | | |

Results are shown by average values in the upper columns and standard deviations in the lower columns (unit: mg/dl).
*There is a significant difference when compared with the maximum blood glucose level of the glucose control group (p < 0.05).

2. Glucose AUC (see Table 8 and FIG. 7)

The glucose AUCs of the test group and the glucose control group were 212.1±92.9 mg/dL·h and 251.6±54.4 mg/dL·h, respectively. Thus, statistically significant difference was not observed between the groups. The glucose AUC of the product control group was 21.1±13.0 mg/dL·h, which was one-tenth of that of the test group or the glucose control group. The results are shown as the average +standard deviation. Further, by a test, the level of significance adopted was not exceeding 5%.

TABLE 8
Glucose AUC

| | Glucose AUC |
|---|---|
| Test group | 212.1 |
| | 92.9 |
| Glucose control group | 251.6 |
| | 54.4 |
| Product control group | 21.1 |
| | 13.0 |

Results are shown by average values in the upper columns and standard deviations in the lower columns (unit: mg/dL · h).

As shown in the results above, in the group in which mice were administered with the product together with glucose (test group), the maximum level of blood glucose was significantly low compared with that of the group in which the mice were administered with glucose only (glucose control group). In addition, the period of time before the blood glucose level reached the maximum became longer, and the period of time before the blood glucose level returned to the level at the time of administration became longer. Therefore, it was confirmed that the product had effects of suppressing an increase in blood glucose level and lowering the increasing rate of blood glucose level when the product was ingested together with glucose under the conditions of this test.

However, since, in comparison of glucose AUC, no difference was observed between the groups, it was suggested that the amount of absorbed glucose was not suppressed by the product. In addition, when the product was administered alone, the blood glucose level was slightly increased. However, the glucose AUC was sufficiently small compared to those of the other two groups. Therefore, it was suggested that the amount of glucose contained in the product was a value not to affect on the results of this test.

As mentioned above, the tea tree mushroom processed product according to the present invention has effects of suppressing an increase in blood glucose level and reducing the absorption of glucose. Therefore, it has been confirmed that the product can be used, for example, as a drug for preventing/treating diabetes mellitus without causing side effects such as hypoglycemia.

### INDUSTRIAL APPLICABILITY

The agent for preventing/ameliorating diabetes mellitus and the functional food for preventing/ameliorating diabetes mellitus according to the present invention are derived from mushroom, i.e., a natural product and have an effect of suppressing an increase in blood glucose level. Therefore, the effect of preventing diabetes mellitus or treating diabetes mellitus can be expected by ingesting the agent in form of a dry powder, a hot-water extract, or a functional food containing them. By using a dried substance of a tea tree mushroom fruit body as a raw material, the above-mentioned effect can be achieved by routinely ingesting the mushroom which is safe and has a good taste.

1. An agent for preventing/ameliorating diabetes mellitus, the agent containing dried tea tree mushroom or a processed substance of the dried tee tree mushroom as an effective component.

2. An agent for preventing/ameliorating diabetes mellitus, wherein the agent contains dried tea tree mushroom or a processed substance of the dried tee tree mushroom and rafuma (yanlong tea).

3. The agent for preventing/ameliorating diabetes mellitus according to claim 1 or 2, wherein the agent further contains at least one component selected from the group consisting of chitosan, water-soluble chitosan, cellulose, hemicellulose, indigestible dextrin, lignin, agar, pectin, glucomannan, sodium alginate, α-lipoic acid, coenzyme Q10, vitamin E, vitamin C, ginkgo leaves, mulberry leaves, β-carotene, lycopene, lutein, astaxanthin, β-cryptoxanthin, catechin, turmeric, blueberry, cranberry, Japanese basil seeds, vitamin B₂, zinc, selenium, and a papaya extract powder.

4. The agent for preventing/ameliorating diabetes mellitus according to any one of claims 1 to 3, wherein the processed substance is a dry powder of tea tree mushroom.

5. The agent for preventing/ameliorating diabetes mellitus according to any one of claims 1 to 3, wherein the processed substance is a capsule, a tablet, or granules of a dry powder of tea tree mushroom.

6. The agent for preventing/ameliorating diabetes mellitus according to any one of claims 1 to 3, wherein the processed substance is a hot-water extract solution of dried tea tree mushroom or a dry powder of tea tree mushroom.

7. The agent for preventing/ameliorating diabetes mellitus according to any one of claims 1 to 3, wherein the processed substance is a dried extract of a hot-water extract solution of dried tea tree mushroom or a dry powder of tea tree mushroom.

8. The agent for preventing/ameliorating diabetes mellitus according to any one of claims 1 to 3, wherein the processed substance is a capsule, a tablet, or granules of a dried extract of a hot-water extract solution of dried tea tree mushroom or a dry powder of tea tree mushroom.

9. A functional food or food material for preventing/ameliorating diabetes mellitus, wherein the functional food or food material contains dried tea tree mushroom or a processed substance of the dried tee tree mushroom.

10. A functional food or food material for preventing/ameliorating diabetes mellitus, wherein the functional food or food material contains dried tea tree mushroom or a processed substance of the dried tea tree mushroom and rafuma (yanlong tea).

11. The functional food or food material for preventing/ameliorating diabetes mellitus according to claim 9 or 10, wherein the functional food or food material further contains at least one component selected from the group consisting of chitosan, water-soluble chitosan, cellulose, hemicellulose, indigestible dextrin, lignin, agar, pectin, glucomannan, sodium alginate, α-lipoic acid, coenzyme Q10, vitamin E, vitamin C, ginkgo leaves, mulberry leaves, β-carotene, lycopene, lutein, astaxanthin, β-cryptoxanthin, catechin, turmeric, blueberry, cranberry, Japanese basil seeds, vitamin B₂, zinc, selenium, and a papaya extract powder.

12. The functional food or food material according to claim 9 or 10, wherein the processed substance is a dry powder of tea tree mushroom.

13. The functional food or food material according to claim 9 or 10, wherein the processed substance is a hot-water extract solution of dried tea tree mushroom or a dry powder of tea tree mushroom.

14. The functional food or food material according to claim 9 or 10, wherein the processed substance is a dried extract of a hot-water extract solution of dried tea tree mushroom or a dry powder of tea tree mushroom.

15. The functional food or food material according to claim 9 or 10, wherein the processed substance is a french-fry of dried tea tree mushroom.

16. The functional food or food material according to any one of claims 9 to 14, wherein the functional food or food material is tea, yogurt, drinkable yogurt, juice, milk, soymilk, alcoholic drinks, coffee, or a sports drink.

17. The functional food or food material according to any one of claims 9 to 14, wherein the functional food or food material is bread or confectionery including baked goods such as pudding, crackers, biscuits, hardtack, cookies, bread, cake, jelly, and rice crackers, Japanese confectionery such as adzuki-bean jelly, frozen dessert, and chewing gum; noodles such as Japanese wheat noodles and buckwheat noodles; fish cakes such as kamaboko (steamed fish paste), ham, and fish meat sausage; a seasoning such as miso (soybean paste), soy sauce, dressing, mayonnaise, and sweetening; a dairy product such as cheese and butter; bean curd; konjac food (alimentary yam paste); a food boiled in soy sauce; gyoza (steam-baked meat pie); croquette; or a salad.
